(19) United States
(12) Patent Application Publication
(10) Pub. No.: US 2010/0073918 A1
(43) Pub. Date: Mar. 25, 2010

(54) Louse-Catching Tweezers

(76) Inventor: Vartan Shaljian, North Hollywood, CA (US)

Correspondence Address: LARRY D. JOHNSON, P.O. BOX 470277, CELEBRATION, FL, 34747, US

(21) Appl. No.: 12/443,675

(22) PCT Filed: Oct. 1, 2007
(86) PCT No.: PCT/US07/80124

§ 371 Date: Mar. 30, 2009

(30) Foreign Application Priority Data
Sep. 30, 2006 (CN) 200620108407.7

(51) Int. Cl. A61B 17/30; B25B 9/00; B25B 23/18

(52) U.S. Cl. 362/120; 294/99.2; 362/157

(57) ABSTRACT

This device comprises a manually operated, battery powered flashlight positioned between and connected to a pair of specially configured tweezers. The shapes of the ends of the tweezers are configured to permit removal of parasites such as lice and ticks. The device itself is configured and sized to be manipulated by only one hand of a user. The ends of the tweezers are shaped to comprise a vertical angle, α=35°+/−3° , and an inward angle, β=80° . These ends, so angled, form a cuspate, grasping mouth, which mouth further defines a lunule hole. The combination of angles further permits a user to firmly press the device against the skin of a host infected with a parasite without puncturing the skin of the host, and also enables the user to firmly position the tweezers around the parasite. The tweezers comprise a pair of generally parallel arms, each of which further defines a through hole that enable a user to more easily grasp and manipulate the tweezers. The flashlight is configured and positioned to shine its light upon the cuspate mouth, thereby illuminating a targeted parasite.

This utility application relates to a tool for removing certain parasites from the skin of humans or pets, without the user having to contact the removed parasite with the user's skin.

It is common for people to own pets. It is unfortunately not uncommon for those pets to bear and host parasites which feed off of hosts after attaching themselves to the host and then boring into the skin far enough to be able to such the host's blood. Such parasites include, but are not limited to, ticks or lice.

It is important to the health of the host to promptly find and remove these parasites: The parasites often transmit diseases to their host, and/or may replicate and thereafter spread their population to other pets, or to humans with whom they frequently come into contact.

It is not only extremely unpleasant, but often difficult or impossible, for a person to safely remove such parasites. Typically, either a person grasps the parasite with the user's fingers, or the person uses a pair of ill suited tweezers or other device, and attempts to remove the parasite. This often crushes the parasite during removal, releasing blood within the parasite, as well as other fluids therewithin. Such contact by a user may cause the host, the user, or both host and user to become infected, diseased, or further infested with parasites.

Avoiding physical contact between a user's skin and a parasite being removed therefore practically eliminates these dangers. The person removing the parasite also eliminates coming into contact with blood released by the removed parasite, or from any wound of the host. It still further eliminates the proximity of a person removing the parasites from parasites that may be “riding” on the host, but not yet become attached.

There is therefore a need for a tool designed to enable a user to remove such harmful and potentially disease carrying parasites from their hosts without the user contacting the parasite with the user's skin. This prevents disease transmission and decreases the chance that parasites on the pet be acquired by the owner.

It is therefore on object of the device disclosed herein to overcome the disadvantages of manually removing parasites from a host, for example a pet.

The device of the disclosure provides a device to enable such parasite removal.

### DRAWINGS

FIG. 1 is a side sectional view of a device of the disclosure.

FIG. 2 is a partial front view of a device of the disclosure, from the position denoted as “F.”

FIG. 3 is a side elevation of a device of the disclosure, both sides being identical.

FIG. 4 is a top view of a device of the disclosure. The bottom of the device is similar in profile to the top view.

### DETAILED DESCRIPTION

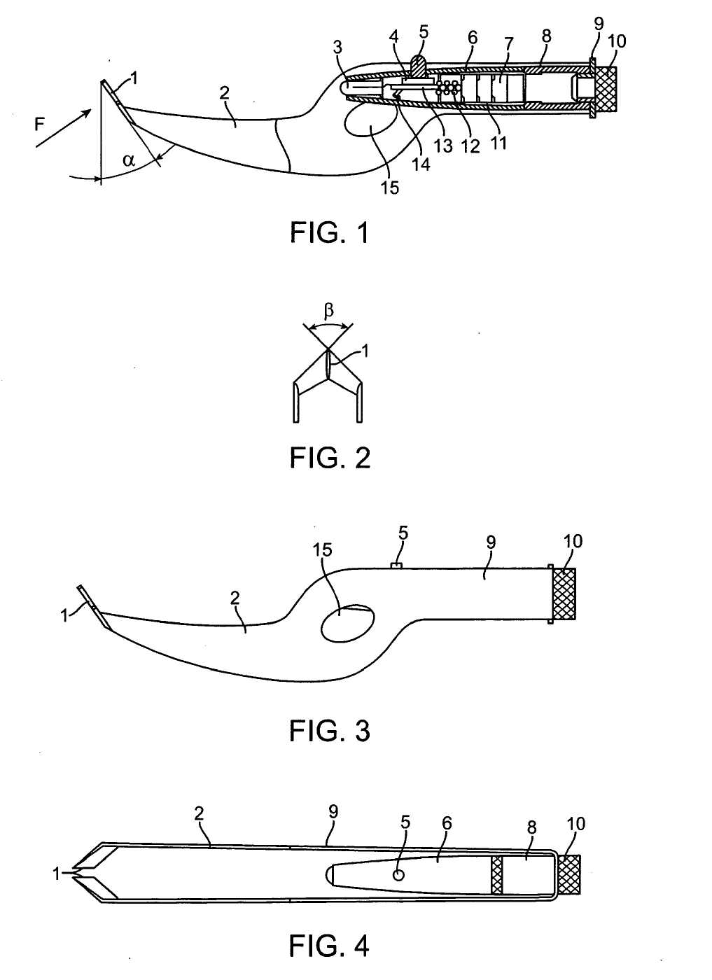

As depicted in FIGS. 1-4, a preferred device according to the disclosure comprises a pair of generally parallel arms joined together in a “U”-shape 9. In a preferred embodiment, the generally parallel arms comprise stainless steel 1 mm thick. A through hole 15 is defined in each of the generally parallel arms, at which through holes 15 a thumb and opposing finger of a user's hand may be positioned. Thus positioned, a user may pinch together the generally parallel arms of the device.

Referring now to FIG. 2, the two generally parallel arms extend forward from their joined “U”-shaped end. The free end of each arm is bent both vertically and inwardly to create a cuspate mouth. The vertical angle of the mouth is α=35°+/−3° . The inward angle of the cuspate mouth is α=80° . This combination of vertical and inward angles forms a pair of grasping or clipping ends 2. Said ends 2 themselves define a cuspate hole 1, which hole enables the grasping of a parasite without crushing or breaking it. This cuspate mouth thereby avoids causing said parasite to release its own internal fluids onto the skin of the host, or onto the user, as happens when such a parasite is crushed or broken.

A flashlight or lamp is coupled to the U-shaped end between the generally parallel arms 9 whereby light emitted from the lamp is aimed at the mouth 1, thereby enabling the device's user to more clearly see a parasite to be grasped or after it has been grasped and removed from a host. The lamp comprises a connecting cap 10 by which the generally parallel arms are connected to the lamp; a front shell 6 removeably threaded to a back shell 8, the back shell further comprising reticulate veins for connection and disassembly. Three button batteries 7 are disposed within an insulating cover 11, which cover is located within the front shell 6.

The front shell 6 further comprises a conical shape enclosing an LED 3 therewithin, the LED 3 comprising an electrical connection to an ON/OFF switch 5. The switch 5 and LED 3 are electrically connected to the batteries 7 by means of springs 12, 14, and circuit board 13. An electrical loop is thereby created which comprises batteries 7, back shell 8, front shell 6, springs 12, 14, switch 5 and LED 3.

What is claimed is:

1. A lighted tweezers device for removing parasites comprising: two substantially parallel clipping arms, each arm comprising a free end and a joined end, which arms together define a “U”-shape, the free end of each arm defining a vertical angle, each said arm further defining an inwards angle, which two non-joined ends of the arms may be displaced by one hand of a user to define a cuspate mouth from the arms; and
a spotlight coupled to and positioned between the joined ends of the clipping arms, wherein the light of the spotlight shines on the cuspate mouth of the tweezers.

2. The device according to claim 1, wherein the vertical angle is an angle α=35°±3° relative to a vertical plane.

3. The device according to claim 1, wherein the inwards angles are angles β=80° .

4. The device according to claim 1, wherein the cuspate mouth defines a lunule hole.

5. The device according to claim 1, wherein a through hole is defined within each of the substantially parallel arms.

6. The device according to claim 1, wherein the spotlight comprises a connecting cap by which cap the joined ends of the clipping arms are coupled to the spotlight, the spotlight further comprising a front shell into which an LED is positioned, the spotlight still further comprising an electrical circuit configured to illuminate the LED, the circuit comprising batteries, a back shell, a front shell, springs, a circuit board and a switch, wherein when the LED is illuminated, light from the LED illuminates the cuspate mouth of the tweezers.
